(19) United States
(12) Patent Application Publication
(10) Pub. No.: US 2016/0198984 A1
(43) Pub. Date: Jul. 14, 2016

(54) Nanocellulose and Nanocellulose Composites as Substrates for Conformal Bioelectronics

(71) Applicant: The Government of the United States of America, as represented by the Secretary of the Navy, Washington, DC (US)

(72) Inventor: Michael A. Daniele, Raleigh, NC (US)

(21) Appl. No.: 14/984,437

(22) Filed: Dec. 30, 2015

Related U.S. Application Data
(60) Provisional application No. 62/101,141, filed on Jan. 8, 2015.

(51) Int. Cl. A61B 5/145; A61B 5/00

(52) U.S. Cl. CPC A61B 5/145; A61B 5/683; A61B 2562/164

(57) ABSTRACT

Described herein is a materials system for electronic device substrates made of nanocellulose and nanocellulose composites that can be transferred to biological tissue while carrying electronic devices. These electronic device substrates are suitable for thin-film electronic devices to adhere and conform to a biological surface, such as human, plant or animal tissue.

### CROSS-REFERENCE TO RELATED APPLICATIONS

This Application claims the benefit of U.S. Provisional Application 61/101,141 filed on Jan. 8, 2015, the entirety of which is incorporated herein by reference.

### STATEMENT REGARDING PRIOR DISCLOSURES BY THE INVENTOR

A prior disclosure, Daniele et al., “Sweet Substrate: A Polysaccharide Nanocomposite for Conformal Electronic Decals,” Advanced Materials, first published online on 3 Dec. 2014, DOI: 10.1002/adma.201404445, was made by the inventor with other named authors. Those other authors who are not named as inventors of this patent application were working under the direction and supervision of the inventor, Dr. Daniele.

### BACKGROUND

Applications for bio-compatible electronics are many, including embedded medical monitoring, diagnostic, treatment, programmable drug dosing devices, tracking, communication, motion monitoring and motion control devices. Substantial efforts have been directed toward developing new solution-processed organic and polymer semiconductors, conductors, and dielectrics that will work in flexible and conformal systems; however progress in biocompatible electronics is also driven by new substrate materials and processing methods due to the intimate contact that the electronic substrate must have with the biological system. It is expected that such processing methods and materials will play an essential role in achieved flexible, stretchable and soft electronic systems. Accordingly, the industry is continually looking for new methods of substrate fabrication and substrate patterning for both performance gains and cost reductions.

Conformable electronics benefit from dielectric substrates that are both thermally and chemically robust, have high mechanical strength and flexibility, and can be manufactured on “larger-than-wafer” scale, such as roll-to-roll, inkjet, or transfer printing. Although there are many approaches to forming flexible substrate layers, they typically fall into one of two categories: a single thick layer of a biopolymer, most commonly silk or gelatin, [Kim, D. H., et al., “Epidermal Electronics.” Science, 2011. 333(6044): p. 838-843. Kim, D. H., et al., “Flexible and stretchable electronics for biointegrated devices.” Annu Rev Biomed Eng, 2012. 14: p. 113-28] or multiple layers of an engineering polymer [Salvatore, G. A., et al., “Wafer-scale design of lightweight and transparent electronics that wraps around hairs.” Nature Communications, 2014. 5, Article No. 2982 and Kaltenbrunner, M., et al., “Ultrathin and lightweight organic solar cells with high flexibility.” Nature Communications, 2012. 3, Article No: 770]. In the case of the simple, single-layer biocompatible substrates made of silk or other protein, the inherent batch variation, low thermal and chemical stability, high aqueous swelling, and rapid dissolution are problematic for electronic device fabrication and extended operational lifetimes. In the case of devices which use a single layer substrate, large thicknesses are required for defect mitigation to ensure high device yield. This required layer thickness typically requires long processing times and limits the flexibility devices. Substrates formed with engineering polymers use a single layer of a polymer substrate attached to a rigid surface for electronic device fabrication that is subsequently peeled away. Plastics-based substrates are most notably limited by solvent swelling and degradation when exposed to conventional solution processed electronic device fabrication. Organic solvents will swell, if not fully solubilize, standard engineering polymers. This limits device processing when using traditional photolithography during conventional manufacturing, making it difficult to perform alignments of transistor components across typical substrate widths up to one meter or more. Traditional photolithographic processes and equipment may be seriously impacted by the substrate's maximum solvent resistance, dimensional stability, solvent swelling, and all key parameters in which plastic supports are typically inferior to rigid inorganic substrates. Significantly, the plastic substrates used in conformal electronics have high oxygen and water barrier properties, which is not optimal for a device being adhered to biological tissue. The barrier properties of the plastic films may be beneficial for the attached electronics; however, it can be damaging to the biological tissue and would also hinder the passage of biochemicals that may be of interest for an electronic biosensor.

Biologically-derived materials, such as silk or fibroin, have potential in niche biomedical applications but tend to rapidly degrade, have poor gas barrier properties, and are costly to manufacture; moreover, at the “conformal” electronics scale the inherent softness of the bio-derived materials results in undue stress on the integrated electronics resulting in strain-induced failures. Plastic materials are great for conformal electronic applications that require optimized barrier properties and do not need chemical interaction between the electronic devices and underlying biological tissue.

Accordingly, a need exists for a high quality, flexible and dielectric substrates that can be formed into thin films for conformal adherence to biological tissue and permit the passage of chemical signals from the tissue.

### BRIEF SUMMARY

In one embodiment, a conformable device includes a biomaterial substrate comprises a laminate of a film of nanocellulose and a water-soluble support. This can include an electronics package mounted to the nanocellulose film, which is no more than 10 microns thick.

In further embodiments, the soluble support is pullulan and/or the nanocellulose is nanofibril cellulose.

### BRIEF DESCRIPTION OF THE DRAWINGS

FIG. 1 is a schematic view of an exemplary nanocellulose substrate supporting a conformal electronics package on top of skin, in which the chemical and physical biosignals can be passed through the substrate to the electronics package.

FIG. 2 is a schematic view of an exemplary transferable electronic circuit (having a thickness ranging from nm to μm) with a nanocellulose film supporting an electronics package on top of a water-soluble sugar layer. The water-soluble sugar layer can be dissolved and the nanocellulose substrate with electronics package can be transferred to the biological tissue like a decal.

FIG. 3 is a schematic view of an exemplary transferable electronic circuit with a nanocellulose film supporting a planarization layer and electronics package on top of a water-soluble sugar layer. The water-soluble sugar layer can be dissolved and the nanocellulose substrate with electronics package can be transferred to the biological tissue as like a decal. The planarization layer is made of an engineering polymer suitable as a substrate on which electronic devices can be fabricated.

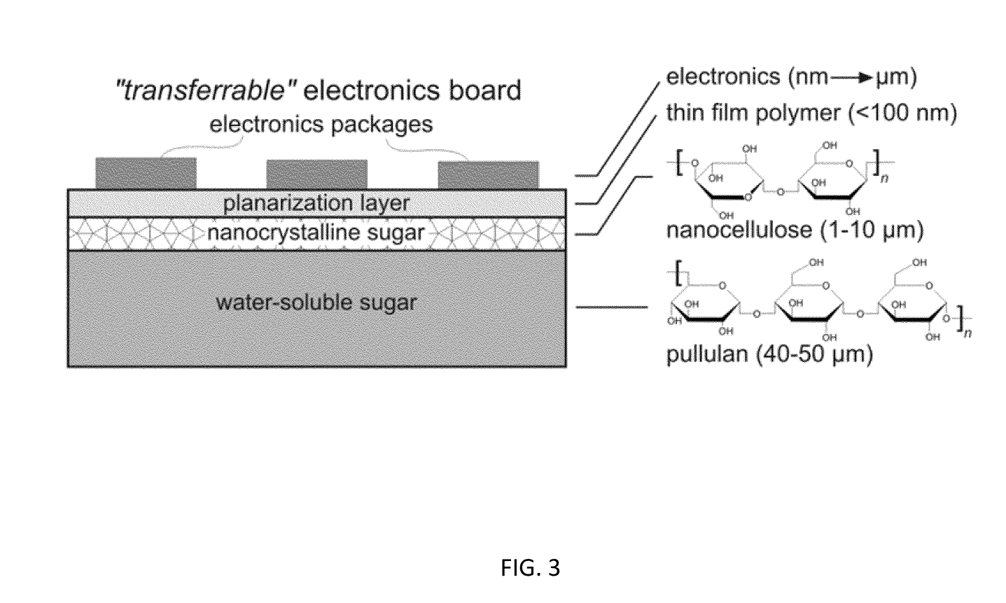

FIGS. 4A and 4B show embodiments of a thin-film substrate composed of nanocellulose that has long fibers.

FIGS. 5A and 5B show conductive traces fabricated on nanocellulose substrates are transparent and flexible.

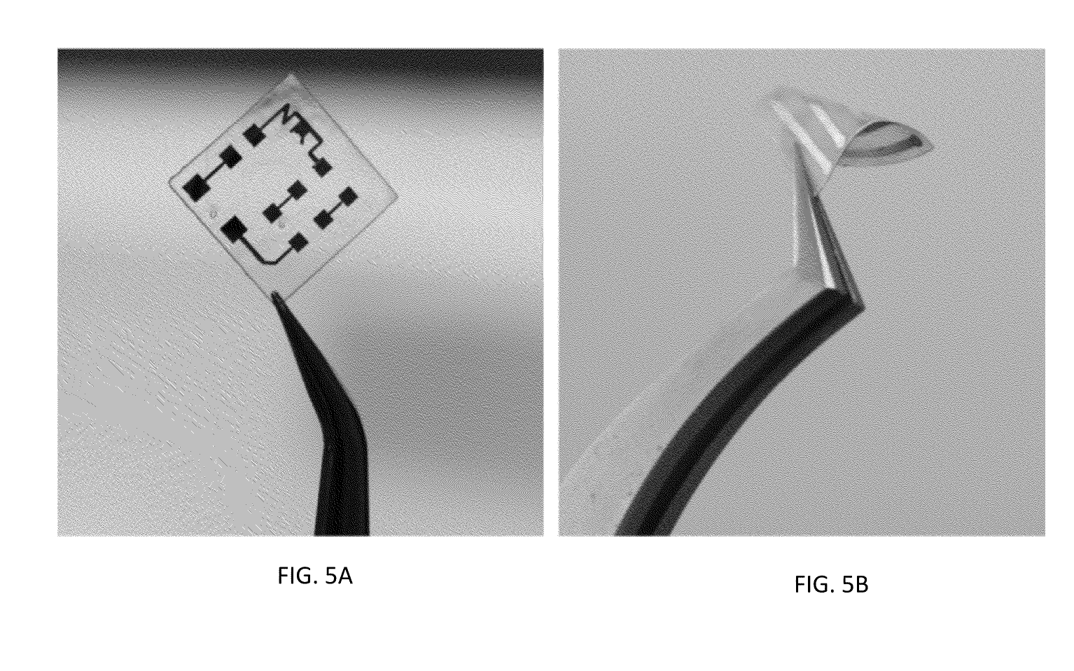

FIGS. 6A and 6B show optical transmission spectra of PCBs for nanocellulose substrates at different distances from the detector and hydration of samples at constant distance from the detector (10 cm).

FIG. 7A shows the Paddington cup used for water vapor transmission rate testing and FIG. 7B shows the water vapor transmission at 23° C., 10% RH (circles) and 37° C., 90% RH (squares), n=3 for nanocellulose substrates. The water vapor transmission rate (WVRT) values ranged between 800-1000 (g·m-2·d-1) were calculated using the exposed surface area of the sample, as follows: WVTR=sample weight change/(area×time).

FIG. 8 shows the weight loss curve (line with square markers) and derivative weight loss curve (plain line) of the nanocellulose composite substrates. Bi-modal decomposition peaks appear at 288° C. and 366° C., which can be attributed to the decomposition of nanofibril cellulose and pullulan, respectively.

FIG. 9 shows the operation of an electronic device on a nanocellulose composite substrate before transfer to a biological tissue.

FIG. 10 shows the transfer of a nanocellulose substrate carrying conductive traces and conforming to the ridges of human skin.

FIG. 11 shows the operation of an electronic device with nanocellulose composite substrates after transfer to a biological tissue using a decal embodiment.

FIGS. 12A through 12D show the operation of an electronic device with a nanocellulose substrate before and after transfer to a biological tissue.

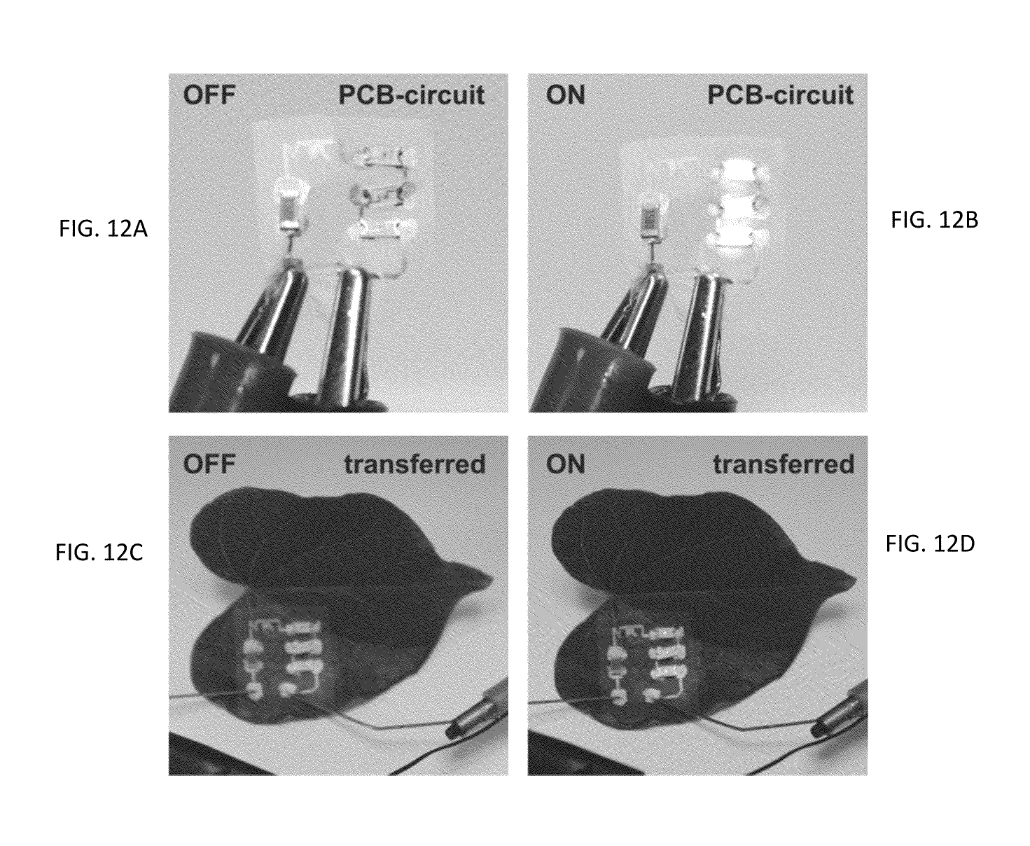

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, nanocellulose refers to a crystalline or semi-crystalline phase of cellulose in which one dimension is less than 100 nanometers.

As used herein, nanofibril cellulose (NFC) refers to nanocellulose where a majority of the fibers have dimensional aspect ratios of 10 or greater, i.e., the fibers have a length at least ten times greater than their diameter.

As used herein, the term nanocellulose composites refers to a combination of nanocellulose and other polymers (either covalently attached or physically blended), which results in a composite material.

Overview

Described herein is a materials system for electronic device substrates made of nanocellulose and nanocellulose composites that can be transferred to biological tissue while carrying the desired electronic devices. These electronic device substrates are suitable for thin-film electronic devices to adhere and conform to a surface, such as epidermal or skin-worn biosensors. The nanocellulose and nanocellulose composites have high thermal and chemical stability, so conventional electronic device fabrication can be conducted. The nanocellulose and nanocellulose composite substrates are well-suited for electronics attached to the skin or other biological tissue because they are bio-compatible, breathable (oxygen permeable), wicking (high water permeation) and will transmit important biochemical signals from the tissue to an attached bioelectronics sensor or device. Preferably, such conformal devices are free of materials that would serve as irritants and/or toxins when in contact with human skin.

A thin nanocellulose or nanocellulose composite film which carries a thin-film electronic device or series of electronic devices and can be adhered to biological tissue (such as skin) by static forces and conforms to the complex surface terrain of the biological tissue.

Particular nanocellulose and nanocellulose composites are designed to provide optimal material functionality for supporting thin-film electronics that can be adhered to biological tissue. The nanocellulose or nanocellulose composite substrate can include a thin-film of nanocellulose (<10 microns) or a thin-film of nanocellulose (<10 microns) laminated to a secondary backing, in which a conventional thin-film semiconductor, polymer or organic electronic device is fabricated on-top, optionally without other layers or materials in the substrate. The nanocellulose provides mechanical support to the carried electronic devices and act as the ultimate substrate for the devices when attached to a biological tissue; whereas, the secondary backing layer in the case of the nanocellulose composite is designed to provide mechanical strength during the fabrication process and be dissolved away before ultimate adherence of the nanocellulose thin-film and electronic devices to the biological tissue, and can facilitate transfer of the nanocellulose in the manner of a decal.

Exemplary Configurations

A first example system is now described. First nanocellulose fibers are provided, and can be prepared from wood pulp by mechanical homogenization. Then the nanocellulose fiber is brought to an appropriate concentration in water, typically by dilution, and cast into a thin-film. An electronic device (such as a simple conductive trace) is fabricated on-top of the nanocellulose thin-film with conventional device fabrication techniques. Due to conformal and static adherence, the nanocellulose carrying a thin-film electronic trace is transferred to a surface, such as human skin. The thin-film electronic traces act as bio-electrodes and the nanocellulose thin-film is a mechanical support and adherence layer.

A second example system is now described. First, a water-soluble polysaccharide film is formed. This film should be of significant rigidity and gas barrier properties to allow for subsequent solution-processing of nanocellulose and electronic devices on-top. It was found that a suitable water-soluble polysaccharide film could be formed by casting of pullulan sugar from an aqueous solution. Nanocellulose fiber (NFC) is prepared from wood pulp by mechanical homogenization. The nanocellulose fiber is diluted in water and cast into a thin-film upon the pullulan sugar film. This nanocellulose film will perform as the ultimate electronic substrate, once the water-soluble transfer backing is removed. An electronic device is fabricated on-top of the nanocellulose thin-film with conventional device fabrication techniques. In an one possible iteration, surface-mount resistors and light-emitting diodes are fashioned into a circuit. Electrochemical cells, transistors or many other more or less complicated circuits can be fabricated on the nanocellulose to comprise a biosensor. The construct is placed on the surface of a water bath and the pullulan base layer is dissolved. The remaining nanocellulose film, carrying the electronic device is transferred to biological tissue.

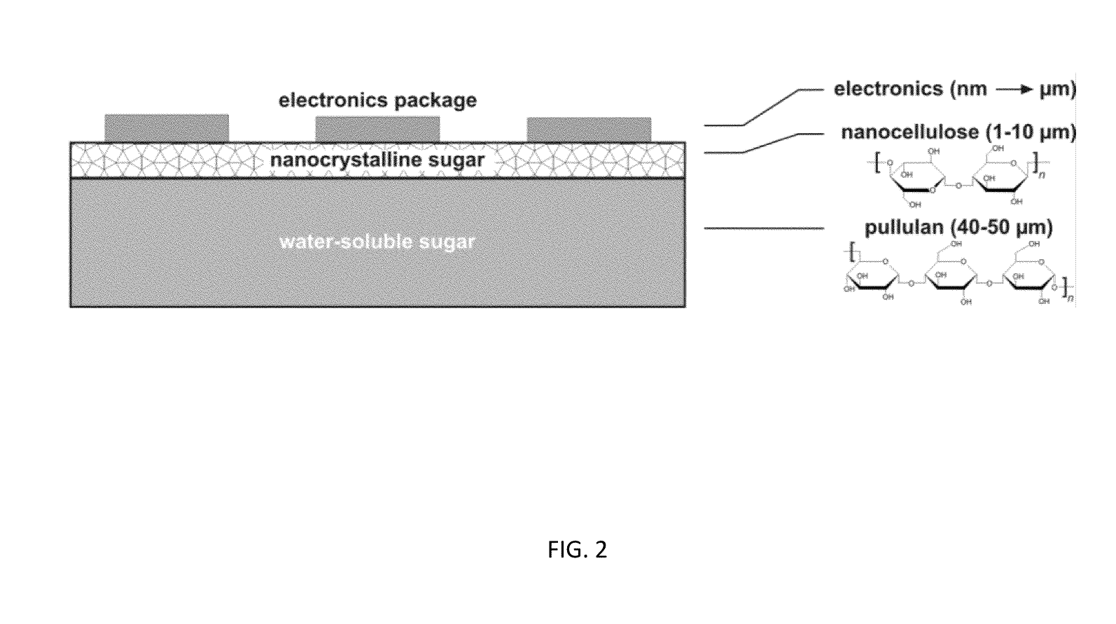

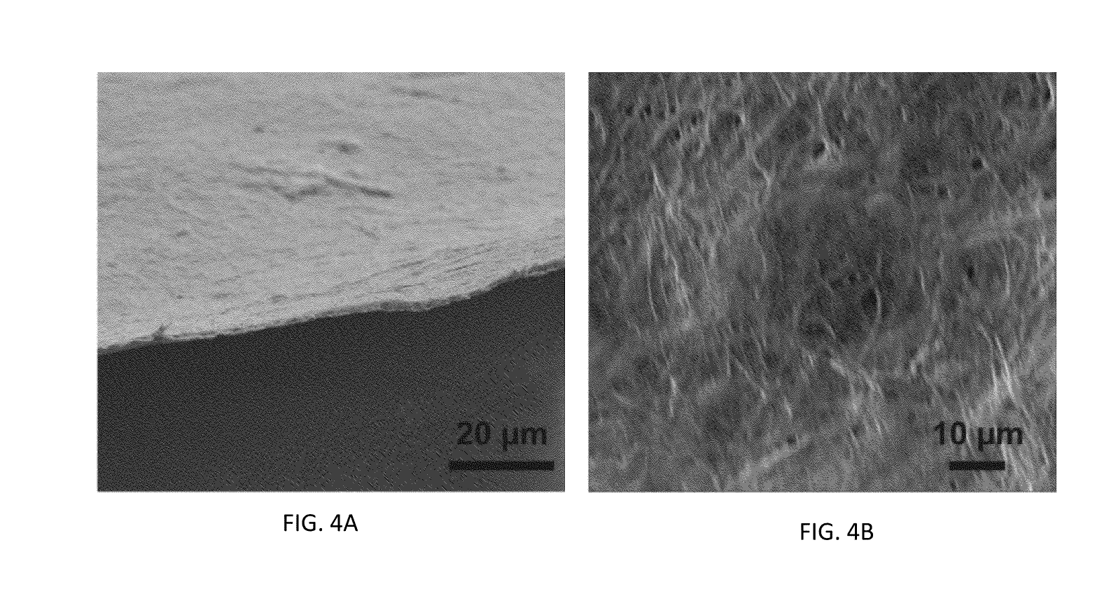

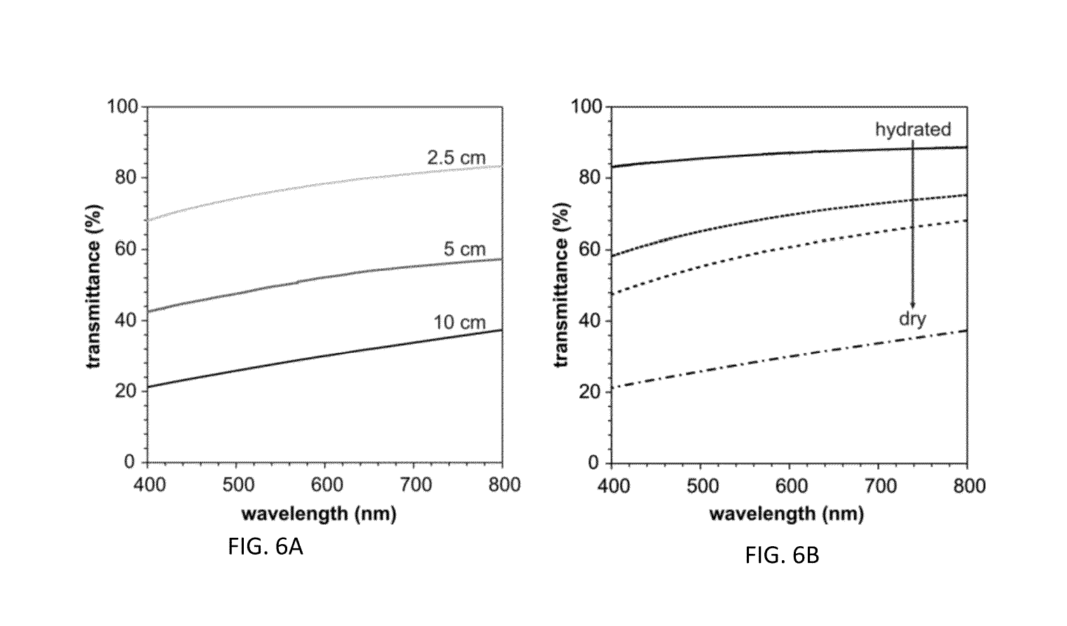

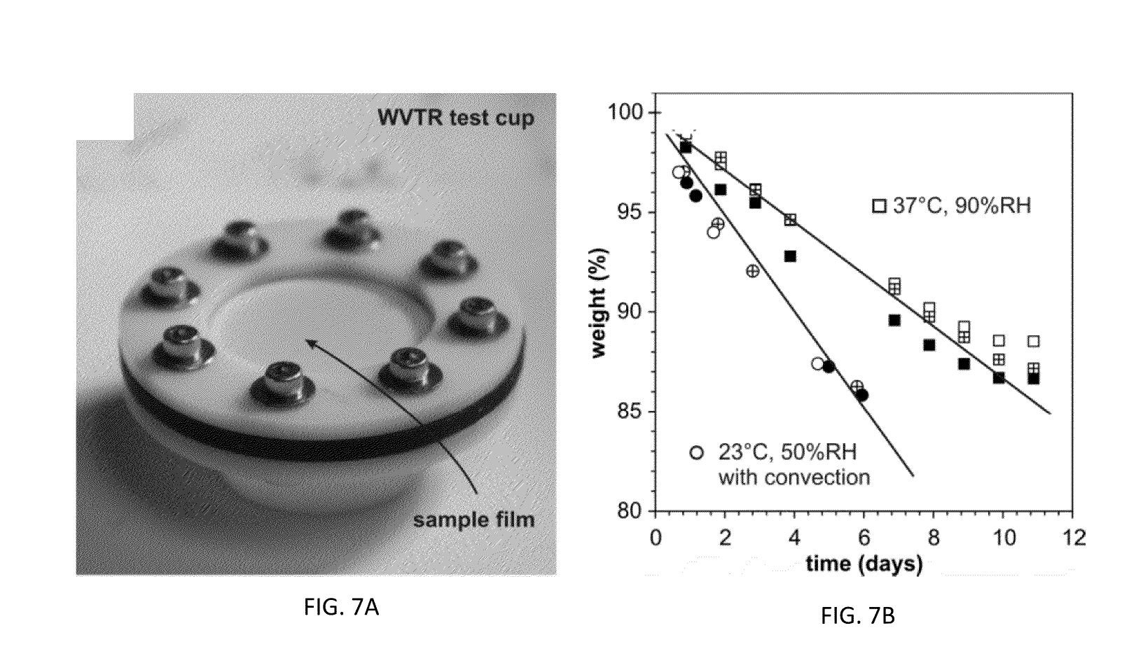

In this iteration, a nanocellulose composite substrate has been used to transfer the electronic devices onto a biological tissue by fabricating a simple decal. The nanocellulose composite substrate was a bi-layer laminate composed of a water-soluble polysaccharide coated in a nanocellulose thin-film (FIGS. 2 & 3). The water-soluble polysaccharides may include pullulan, sucrose, fructose, etc. The nanocellulose layer can be composed of nanocellulose with varying crystal size and shapes, from nanorods to nanofibers (FIGS. 4A and 4B). When the water-soluble polysaccharide is dissolved away, the remaining thin film of the crystalline polysaccharide provides a support for any electronic device built on top and has the requisite mechanical properties to conform to a carrier substrate without cracking, delamination or fracture of the electronic elements. The resulting nanocellulose composite decal has high optical transparency (>80% from 400 to 750 nm) (FIGS. 6A and 6B), ruggedness, and a relatively low thermal expansion, as well as high water-vapor transmission (FIG. 7).

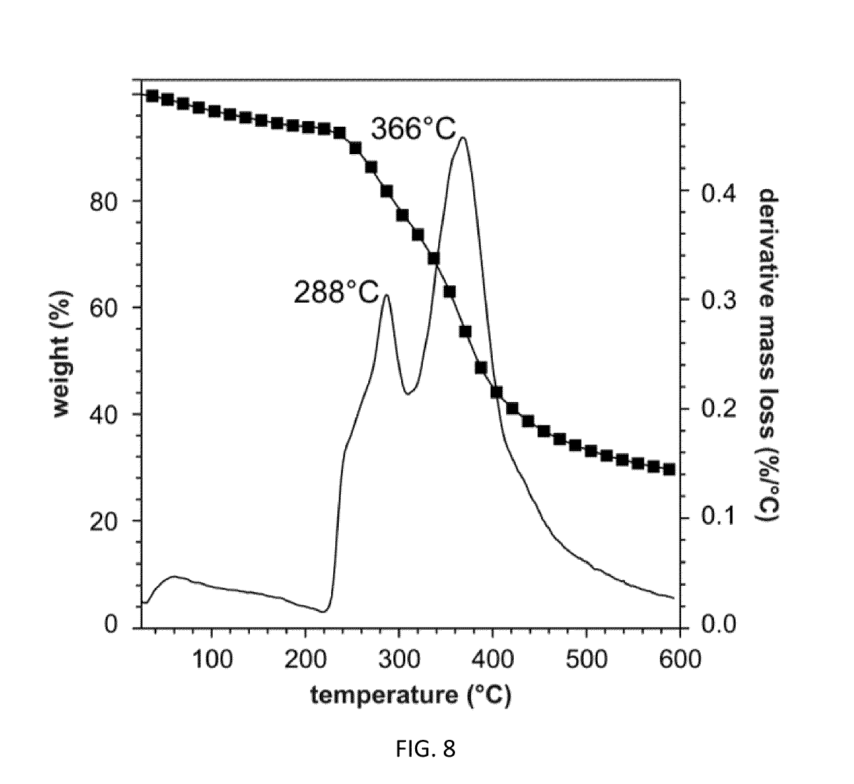

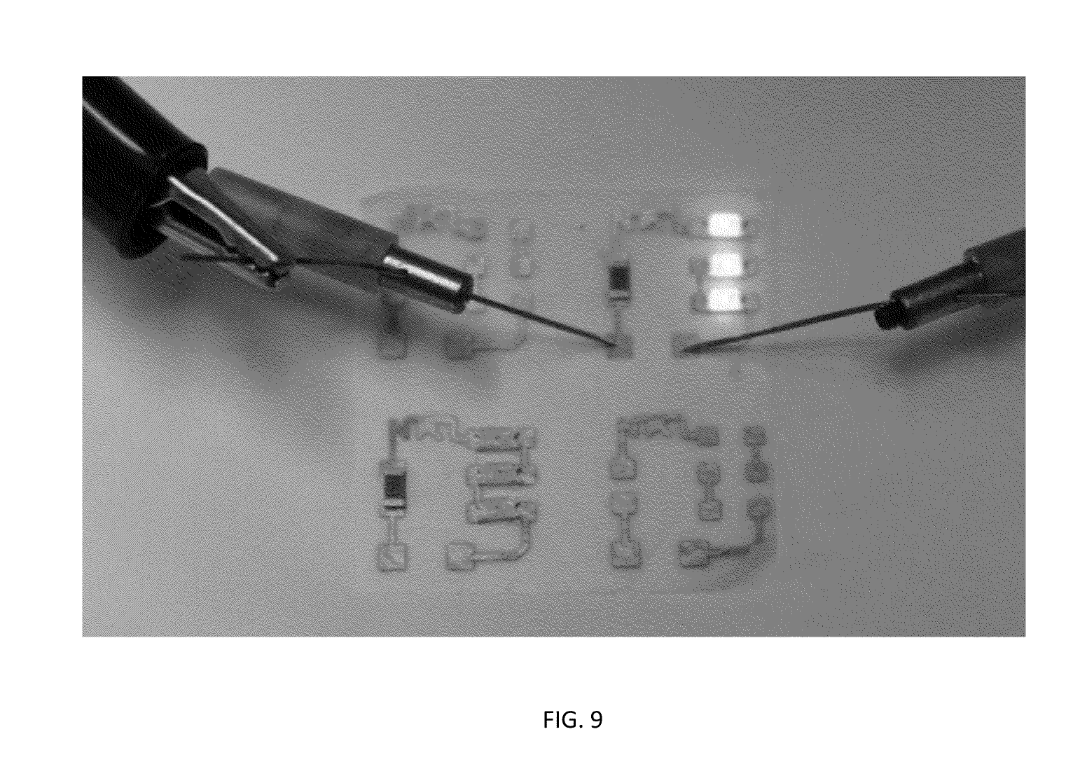

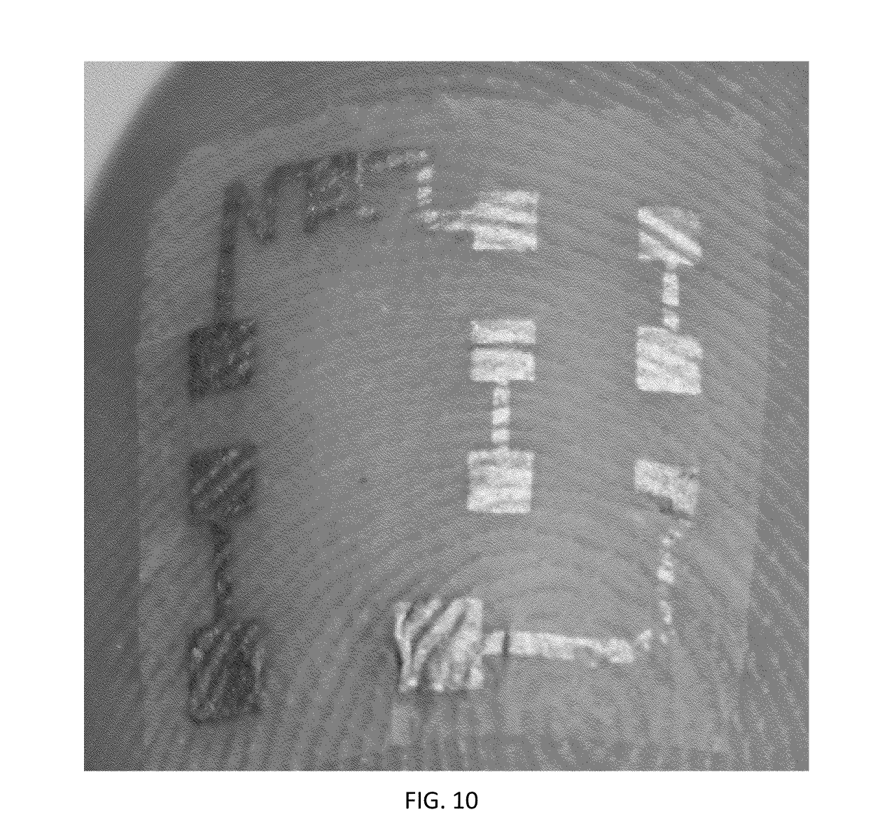

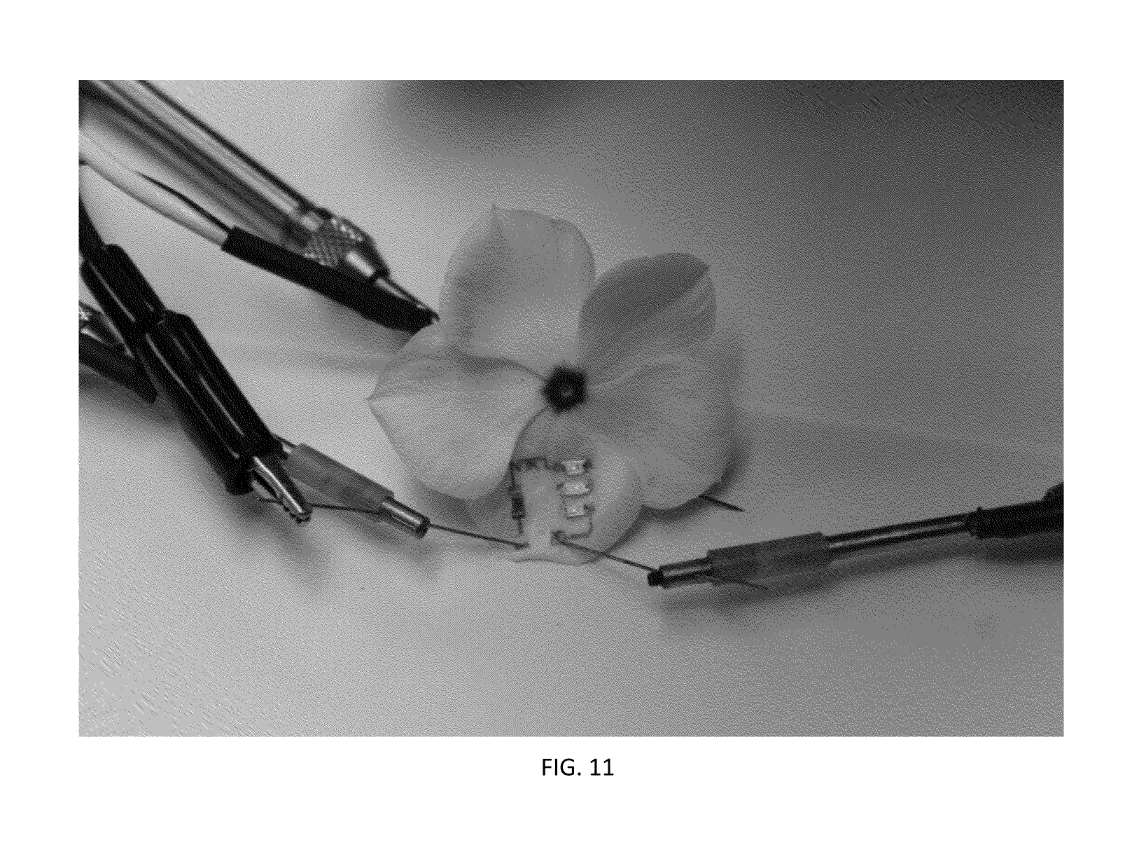

A number of electronic device structures can be made with the functional layers described above. Preferably, the critical interfaces are created and have solution processable or depositions processes below critical degradation temperature of the nanocellulose and nanocellulose composites (FIGS. 8A and 8B). The fabricated nanocellulose substrate decals carry the electronic devices and function properly (FIG. 9). The entire construct can be transferred to biological tissue (FIGS. 10, 11, & 12). Once transferred to the biological tissue, the electronic devices properly function and the nanocellulose substrate acts as a conventional circuit board (FIGS. 11 & 12).

By casting from an aqueous pullulan solution, it is believed that swelling and limited diffusion between the pullulan and NFC transfer occurs, which results in strong intermolecular bonding between the layers of the laminate. Casting from alcohol-based suspensions was attempted, however use of an alcohol-based suspension resulted in poor adherence between the laminate's layers and heterogeneity across the laminate's surface. This is attributed to the poor solubility of pullulan in organics.

A composed NFC-pullulan decal was placed in a drying oven between glass plates to obtain low surface-roughness and uniform thickness. In a similar fashion to conventional printed circuit boards, a series of gold traces were evaporated onto the NFC-pullulan decal. The resultant polysaccharide circuit boards (PCBs) were composed of an NFC transfer of 5 μm and a pullulan support of 50 μm. Suitable NFC layer thickness can range from, for example, about 1-10 μm with pullulan layer thickness from about 40-50 μm.

The NFC was produced by mechanical homogenization of bleached wood pulp, resulting in NFC with diameters of ≈10-20 nm and lengths of ≈500-1000 nm. After drying, the NFC decal transfer is insoluble because strong inter-fibrillar interactions are formed, including mechanical entanglement and hydrogen bonding between surface hydroxyl groups. Pullulan is a maltotriose polysaccharide. It is a good film-forming polysaccharide, and as an edible, mostly tasteless polymer, the chief commercial use of pullulan is in the manufacture of candy films, food additives, and oral/transdermal drug delivery agents. Pullulan was selected because it has a high aqueous solubility, but once dried; the pullulan decal support is mechanically, chemically and thermally robust enough for subsequent electronic device fabrication. These polysaccharides are convenient not only for their biocompatibility, but also for their aqueous processing and ability to withstand treatments with most organic solvents utilized during the electronics fabrication procedures.

For transfer to the epidermal surface, the PCB was simply floated in a container of water, and after the pullulan decal support dissolved, the remaining NFC decal transfer with conductive traces was placed on a human fingertip. A typical wet-slide decal relies on a dextrose residue to bond the decal transfer to a surface, the NFC decal transfer adheres to a surface by static van der Waals forces. A film thinner than about 10 μm is expected to have conformal adherence to epidermal surfaces without adhesives, otherwise adhesives may be used for adhesion.

Many of the functional criteria for conformal electronics substrates are dependent on mechanical properties. The mechanical properties of nanocellulose and nanocellulose composites vary depending on source and sample preparation. Significant deciding factors are crystallinity and crystal shape, e.g. pulp-derived cellulose nanocrystals exhibit an elastic modulus greater than 125 GPa but hemp-derived nanofibrils exhibit an elastic modulus of 33 Gpa. Standard tensile tests on the PCB and constituent polysaccharide components show the significant effect of the NFC's inherent strength on the resultant mechanical properties of the PCB. The slope of the linear portion of the stress-strain curve was determined as the elastic modulus. The elastic modulus of the PCB was 2.25±0.3 GPa; whereas the elastic modulus of a pullulan and bulk NFC were 0.69±0.1 GPa and 4.95±0.5, respectively. For reference, polyethylene terephthalate (PET), a common substrate for flexible electronic systems, was tested. The resultant elastic modulus of the PET film was 2.43±0.1 GPa. The PCBs exhibit mechanical strength in the range necessary for a viable electronic system substrate. To characterize the electronic properties of the PCBs, the resistance of the conductive traces measured under different strains. The PCBs reached a 5% increase in resistivity under a tensile strain of 1%. Due to the high mechanical strength of the NFC decal transfer, there is a very small range of tensile strain and the conductive traces experience very little deformation. Under compressive strain, the PCB folds and wrinkles, much like a sheet of tissue paper. Nonetheless, the PCBs exhibited a maximum change in resistance of 1.35× the starting value, and this resulted when the PCB was completely folded in half. Other paper-based circuit boards exhibit different conductance under positive and negative folding because there is a significant strain mismatch between the inside and outside surfaces of the thick substrate. To examine a more complex strain of the PCB, samples were radially twisted, i.e. one side of the PCB was held stationary and the other was twisted through 90°. The resistance of a simple linear trace showed little change in resistance with twisting. This result was expected, as the axis of the twist was in line with the trace's long axis. An “L-shaped” trace was also monitored and exhibited increased resistance with twisting. Although the traces showed good adherence with the PCB, a multi-directional and complex strain out of axis resulted in cracking and delamination of the trace, after 60° twist. The consistent resistance under both tensile and compressive strain is beneficial for a conformal substrate that will map to a complex terrain. It can be foreseen that future iterations may easily replace the bulk metal traces with stretchable electrodes and electronic components, and these future design considerations will accommodate greater mechanical deformation and improve the performance of the PCB while conforming to a complex surface.

If other electrode designs and device are to be used with the PCBs, thermal stability is a critical parameter for determining the possible electronic device fabrication techniques that can be employed with the PCBs. The thermal degradation of the PCBs was examined by thermogravimetric analysis. Component polysaccharides showed initial weight loss between 70-80° C., which is attributed to water evaporation. Significant thermal degradation occurred between 280 and 500° C. The PCB can tolerate a much higher processing temperature than recently reported bio-derived substrates for flexible electronics. For example, gelatin or silk substrates exhibit thermal instability and transitions at physiologically relevant temperatures (37° C.). The thermal stability of PCBs is amenable to electron-beam evaporation to generate conductive traces, and it may enable techniques requiring elevated processing temperatures, such as atomic layer deposition, chemical vapor deposition, transfer printing, and/or imprint lithography.

An often overlooked property for electronic device substrates used in biological systems is the water vapor transmission rate (WVTR). Conventional electronics require high vapor and gas barrier properties, but an epidermal or implanted device should permit the transmission of water vapor, oxygen and other compounds to prevent tissue damage Wicking of liquids produced at the biological-electronic interface would also be necessary to prevent delamination, from sweat or detritus build-up. Due to intermolecular hydrogen bonding, nanocellulose films exhibit water vapor and oxygen barrier properties dependent on nanofibril size and chemical pretreatment (as described in A. Dufresne, Nanocellulose: From Nature to High Performance Tailored Materials, De Gruyter, 2012, incorporated herein by reference for disclosing techniques for controlling such properties). Accordingly, WVTR of the NFC decal transfer was investigated by using the Paddington cup method. WVTR was measured at 23° C./10% RH with a 0.5 mm·s⁻¹ forced convection across the surface to simulate epidermal wear and at 37° C./90% RH to simulate implanted wear. The WVTR is dependent on temperature and relative humidity, ranging from a high of 1627±158 g·m⁻²·day⁻¹ at 23° C./10% RH to a low of 781±74 g·m⁻²·day⁻¹ at 37° C./90% RH. The WVTR of the NFC transfer decal is comparable to some common bandages and wound dressings.¹ Conventional polymer films used in flexible electronics have WVTR between 1-40 g·m⁻²·day⁻¹ and could be detrimental to the epidermal or biological surface over long periods. The NFC decal transfers were stable in low and high humidity environments for the duration of the WVTR testing. Under ambient conditions, the PCBs were stable for weeks without any visible or measurable signs of degradation.

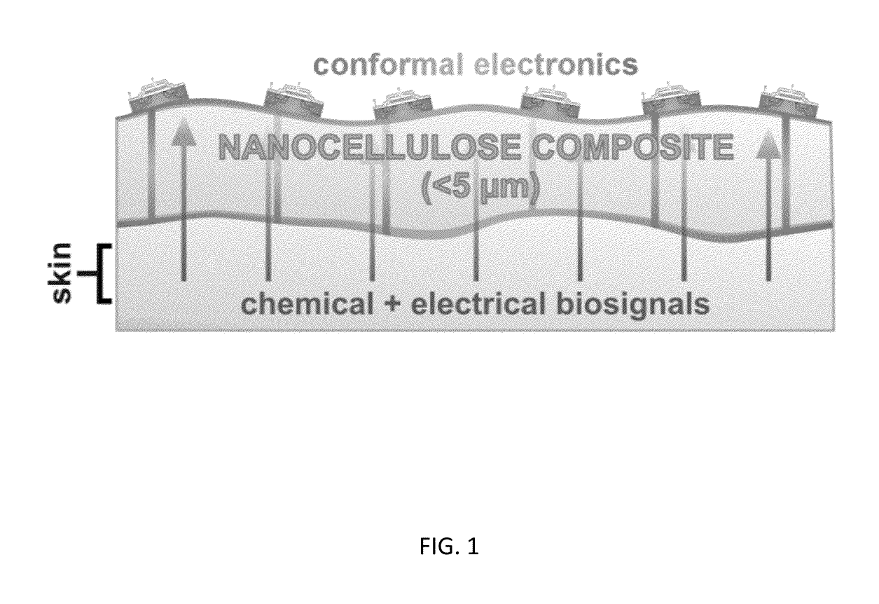

To demonstrate the utility of PCBs as viable substrates for electronic devices, a simple light-emitting diode (LED) circuit was prepared. After preparation of the PCB (cf. FIG. 1), conductive ink was printed onto the PCB via a modified Meyer Rod printing method then surface-mount devices were attached. The printed conductive ink acts as solder on a conventional printed circuit board, and the printed inks are flexible and can be applied in a commercial printing method. The PCB-LED circuits were constructed at the wafer-scale. The “wet-slide” decal transfer was gentle and robust enough to provide for PCB-LED circuit operation on a flower petal. The as-fabricated PCB-LED circuit exhibited expected operation with a turn-on voltage at 5.4 V. The PCB-LED circuit was transferred using the “wet-slide” decal method and operated in conformal contact with the biological surface. The transferred PCB-LED circuit exhibited diminished luminance, attributed to increased contact resistivity during the transfer process and conformation to the biological surface. The PCB-LED circuit showed no degradation during continued operation or with multiple on/off cycles. The PCB showed no cracking or mechanical wear during transfer to the biological surface or operation.

To better analyze the operation of the PCB during and after the decal transfer process, the conductive traces' resistance was monitored as the PCBs were placed in water to dissolve the pullulan decal support, and then the remaining NFC decal transfer was applied to glass rod with a radius of curvature of 1 mm and resistance measured over time. Initially the resistance is stable, and then it begins to increase as the pullulan decal support dissolves and water infiltrates the NFC decal transfer. Once the pullulan decal support is completely dissolved the resistance of the trace stabilized at approximately 1.75× the initial resistance. Finally, the NFC decal transfer is flexed around a glass rod and dried. The resistance returns and stabilizes near the original value. After application of the NFC decal transfer to a glass rod, the samples exhibited a 9% decrease in conductance, on average. The strength and thinness of the NFC decal transfer provided conformal and intimate contact with the glass surface, and it did not result in noticeable delamination of the conductive traces. It was also observed that the NFC decal transfer operated after the initial transfer and subsequent peeling from the glass rod. The chemical modification of the NFC may even further reduce the resistance change during the decal transfer process or under wet operation by changing both electronic characteristics and hydrophobicity.

Suitable NFC fibers may have diameters of less than 100 nm, e.g., as small as 10 nm or less, and fiber lengths of at least 10 times their diameter including lengths of up to or exceeding one millimeter.

In summary, a polysaccharide circuit board was developed for conformal electronic decals by composition of a nanocellulose-pullulan laminate. The PCB was simply prepared by casting a thick pullulan (50 μm) decal support onto a nanocellulose thin-film decal transfer (5 μm). The different aqueous solubility of the two polysaccharides provided for selective dissolution of the pullulan support, and the NFC thin-film could be transferred like a water-slide decal. The PCBs exhibit good thermal and mechanical properties, in comparison to conventional soft electronics substrates. The strength of the NFC provided for a thin, robust decal transfer that could adhere to biological surfaces by static forces. The PCBs also displayed high water vapor transmission rates, which is an often overlooked property for any epidermal or biological conformal electronic system. At the biological interface, the electronic substrate must transmit or wick away fluid at the surface to prevent delamination. These laminates provided a practical substrate for patterning conductive traces and fabricating simple circuits.

As a complete package, the PCBs performed admirably under mechanical, thermal and chemical stresses providing a decal for conformal electronics substrates. This approach could provide a scalable material system for the production of conformal circuit boards and electronic decals; thus, there are a range of applications to explore in which light-weight conformal electronics would benefit from a robust, tunable substrate, e.g. environmental tags, personal electronic devices, flexible displays, micro-aeronautics and biological sensors. Ultimately, t the use of these PCBs may be expanded by integrating stretchable electrodes and biosensors onto the decals; wherein, the thinness and conformal contact of the PCB attached to the epidermis or other biological substrate will create a scenario in which the biological tissue becomes an integral part of the electronic device's operation.

Nanocellulose sheet may optionally carry a thin-film electronic device or series of electronic devices; and the resultant product can be adhered to biological tissue by static forces, conforming to the complex surface terrain of the biological tissue. Electronic devices can include several components, for example conductors, one or more types of sensors (for example, a pressure sensor, pH sensor, strain sensor, and/or a sensor for electrodermal activity), a battery, a transmitter and/or receiver of wireless data, one or more antennas, electrodes (optionally configured to transmit electrical energy to the skin), a microprocessor, digital memory, passive and active circuit components, and the like.

Experimental Techniques

Materials:

Nanofibril cellulose was purchased from the U.S. Forest Service Cellulose Nano-Materials Pilot Plant at the Forest Products Laboratory in conjunction with the Process Development Center at the University of Maine (Orono, Me.). The NFC was prepared by ultrafine grinding with a Masuko MKZB15-50J supermass colloider, and the final product was provided as 3% solids NFC slurry. The NFC slurry was diluted to 1% solids and homogenized with an IKA Ultra Turrax (Wilmington, N.C. USA) homogenizer for 20 min. at 20000 rpm, before further use. Pullulan was purchased from V-Labs, Inc. (Covington, La. USA). Conductive silver printing inks and thinner were purchased from DuPontUSA (Wilmington, Del. USA). Surface-mount devices were purchased from Mouser Electronics (El Cajon, Calif. USA). Lumex Standard LEDs (1206 Red) and KAO-Speer thick-film resistors (1206/0.25 W, 15052) were utilized for the PCB-LED circuit. Nanocellulose was dialyzed (MWCO=5,000 Da) against water to remove any impurities resulting from processing. All other purchased chemicals were used without further purification. Deionized water was obtained from a milliQ Nanopure System and exhibited a resistivity of ca. 10¹⁸ ohm-1 cm-1.

Preparation of PCB:

7.85 g of NFC was suspended in 100 mL of water by homogenization for 20 min. The NFC suspension was sonicated for 30 min. to remove air bubbles. The NFC suspension (10 mL) was cast onto a leveled, silicone-coated glass plate and dried at room temperature for 24 h. 5 g of pullulan was dissolved in 100 mL of water, and the solution was filtered through a nylon membrane (0.45 μm). The pullulan solution (10 mL) was cast onto the NFC film in the glass plate and dried in an oven at 65° C. The NFC-pullulan laminates were sequentially cleaned with acetone and isopropanol in an ultrasonic bath for 30 min. The NFC-pullulan laminates were dried in an oven overnight at 65° C. Metal tracks and pads were evaporated onto the NFC-pullulan laminates through an aluminum shadow-mask (Temescal Model FC-2000 E-Beam Evaporator (Livermore, Calif. USA)). Metal tracks and pads were a titanium adhesion layer (10 nm) and gold (100 nm).

Fabrication of PCB-LED Circuit:

Conductive silver ink was printed on the PCBs through a nylon screen by a modified Meyer rod process. Surface-mount devices placed and the conductive ink was cured in an oven at 65° C.

Characterization:

Mechanical properties of the PCBs were investigated using a dynamic mechanical analyzer (DMA-Q800, TA Instruments, New Castle, Del. USA). PCBs were stored in a desiccator overnight to ensure removal of any residual water or cleaning solvent and cut into rectangular samples (8×30 mm). Each sample was inserted into the grips with a 10 mm gauge length. Elastic modulus was investigated by applying a tensile stress at a constant rate of 1 N·min-1. Elastic modulus is reported as the average and one standard deviation of the sample set, n=6. The thermal stability was measured by thermogravimetric analysis (TGA-2900, TA Instruments, New Castle, Del. USA). The samples (≈10 mg) were heated from 25° C. to 600° C. at a heating rate of 10° C.·min-1 and a nitrogen flow of 100 mL·min-1. The WVTR measurements were performed using a circular PCB (5.07 cm2) sealed in an impermeable container containing saturated salt solutions to maintain constant relative humidity. The decrease in weight was measured by a microbalance, and the WVTR was calculated from the slope of weight change versus time. WVTR are reported as the average and one standard deviation of the sample set, n=3. Optical transmission measurements were made with a UV-Vis-NIR Spectrophotometer (Cary 5000, Santa Clara, Calif. USA). Electronic properties were characterized under ambient conditions using a semiconductor parameter analyzer equipped with a 20V/100 mA source-measurement unit (Hewlett-Packard 4155A, Palo Alto, Calif. USA) and a standalone source-measure unit with microprobe station (Model 2600, Keithley Instruments, Cleveland, Ohio USA).

Alternatives

The above-described materials may be varied. Cellulose for preparation of nanocellulose may be obtained from bacterial, tunicate, plant, and/or other biomass sources. One or more different film-forming water soluble sugars can replace the pullulan base layer, however does serve as a good film-forming and oxygen barrier sugar.

Fabrication of the nanocellulose and nanocellulose composites can be done by many methods other than casting and spin coating: silk screening, roll-to-roll, spray deposition, doctor blade, dip coating, etc.

Various device manufacturing processes are suitable for use with the substrates described herein, as long as the technique stays below the degradation temperature of the nanocellulose. Such techniques include evaporative deposition, sputtering, and printing or transfer techniques.

Advantages

The use of nanocellulose and nanocellulose composites for conformal bioelectronics substrates improves upon many substrates currently being used in research. Substrate properties such as water and oxygen permeability are well-suited for applications involving adherence to biological tissue. These properties also make the nanocellulose and nanocellulose composite substrates a good choice for biochemical sensors which would rely on the transmission of biochemicals present at the biological tissue surface.

Nanocellulose and its nanocomposites have exhibited impressive mechanical strength (E≈150 GPa) at low density (ρ≈1.5 g·cm-3), low coefficient of thermal expansion (4-18.5 ppm·° C.-1), tunable optical transparency, tunable gas-barrier properties, while exhibiting dielectric properties (κ≈1.3-2.0) comparable to other substrates for flexible electronics.

Furthermore, conventional electronics fabrication techniques may be used, whereas they can degrade other substrates.

Nanocellulose and nanocellulose composites are cost-effective, ubiquitous biomass products obtainable from renewable resources. They also have high inherent strength, such that film thickness required for conformal adherence without added adhesives (<10 microns) is attainable.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

1. A conformable electronic device comprising:
a biomaterial substrate comprising a laminate of a nanocellulose film and a water-soluble support; and
an electronics package mounted to the nanocellulose film,
wherein the nanocellulose film has a thickness of no greater than 10 microns.

2. The conformal electronic device of claim 1, wherein the water-soluble support is a polysaccharide.

3. The conformal electronic device of claim 2, wherein the soluble support is pullulan.

4. The conformal electronic device of claim 1, the nanocellulose is nanofibril cellulose.

5. The conformal electronic device of claim 1, having a water vapor transmission rate of at least 781 g·m⁻²·day⁻¹ when measured at 37° C. and 90% relative humidity.

6. The conformal electronic device of claim 1, wherein the nanocellulose is nanofibril cellulose.

7. A conformable electronic device comprising:
a biomaterial substrate comprising a laminate of a nanofibril nanocellulose film and a water-soluble pullulan support; and
an electronics package mounted to the nanocellulose film,
wherein the nanofibril nanocellulose film has a thickness of no greater than 10 microns, and
wherein the device has a water vapor transmission rate of at least 781 g·m⁻²·day⁻¹ when measured at 37° C. and 90% relative humidity.

8. A method of making a conformable electronic device, the method comprising:
providing a water-soluble support film;
casting a nanocellulose film on top of the support film; and
fabricating an electronic device on top of the nanocellulose film to obtain the conformable electronic device.

9. The method of claim 8, wherein the water-soluble support film comprises pullulan.

10. The method of claim 8, wherein the nanocellolose film comprises nanofibril cellulose.

11. The method of claim 10, further comprising preparing the nanofibril cellulose by mechanical homogenization of bleached wood pulp.

12. The method of claim 8, wherein the conformable electronic device has a water vapor transmission rate of at least 781 g·m⁻²·day⁻¹ when measured at 37° C. and 90% relative humidity.

13. The method of claim 8, wherein the fabricating of the electronic device includes printing conductive ink onto the nanocellulose film.
